(19) United States
(12) Patent Application Publication
(10) Pub. No.: US 2008/0020460 A1
(43) Pub. Date: Jan. 24, 2008

(54) Method for Storing Melanocytes as a Suspension

(75) Inventors: Bin-Ru She, Cyonglin Township (TW); Chih-Ching Liao, Sinpu Township (TW); Tzu-Pin Shentu, Kaohsiung City (TW); Ying-Jung Yang, Hsinchu City (TW); Yi-Ping Wu, Nantou City (TW)

Correspondence Address: THOMAS, KAYDEN, HORSTEMEYER & RISLEY, LLP, 600 GALLERIA PARKWAY, STE 1500, ATLANTA, GA, 30339, US

(73) Assignee: INDUSTRIAL TECHNOLOGY RESEARCH INSTITUTE, Chutung Town (JP)

(21) Appl. No.: 11/740,403

(22) Filed: Apr. 26, 2007

(30) Foreign Application Priority Data
Jul. 20, 2006 (TW) 95126591
Aug. 30, 2006 (TW) 95132050

(51) Int. Cl. C12N 5/02

(52) U.S. Cl. 435/374

(57) ABSTRACT

The present invention is related to a method for storing melanocytes. The melanocytes suspension of the present invention can be stored at a temperature range from 4° C. to 22.5° C., wherein the storage temperature for baby melanocytes suspension ranges from 4° C. to 15° C. The viability of the cells after 48 hours of storage at such temperature range is at least 71% of that of the pre-stored cells. The storage temperature for adult melanocytes suspension ranges from 10° C. to 22.5° C. The viability of the cells after 24 hours of storage at such temperature range is at least 72% of that of the pre-stored cells. Meanwhile, storage of the melanocytes at such temperature range prevents the aggregation of the melanocytes. The melanocytes suspension can be used for the treatment of leukoderma as well as other conditions that result from the lack of melanocytes.

### RELATED APPLICATIONS

This application claims priority to Taiwan Application Serial Number 95126591, filed Jul. 20, 2006, which is herein incorporated by reference.

This application also claims priority to Taiwan Application Serial Number 95132050, filed Aug. 30, 2006, which is herein incorporated by reference.

### BACKGROUND

1. Field of Invention

The present invention relates to a method for storing adherent cells. More particularly, the present invention relates to a method for storing melanocytes as a suspension.

2. Description of Related Art

Vitiligo, a kind of Leukoderma, is a progressive disease in which the melanocytes are gradually destroyed that causes un-pigmented areas on the skin. It happens in 1-2% of the population. The clinical symptoms of vitiligo are that some irregular white spots appear on skins or mucous membrane, and melanocytes of the affected parts almost disappear. There are many treatments for vitiligo, such as treating with steroid medicines, or skin transplantation. However, skin transplantation is not applicable for large areas, while steroid treatment might cause systemic or local side effects and has limited successful rate.

The most popular treatment presently is phototherapy, such as ultraviolet A (UVA) or narrow-band ultraviolet B (NUVB) treatment, with or without the conjunctive application of photosensitizers, Psoralen e.g. However, phototherapy usually requires 100 to 250 treatments during one to two years of treatment course. Furthermore, in average only 50% of patients show apparent curative response to the therapy. Therefore, another novel treatment has been developed that is transplantation of melanocytes into the vitiligo lesion. This method is more efficacious with the repigmentation usually appearing in a few weeks after the treatment and without apparent side effect. This therapy requires only a few visits to the hospital.

Melanocytes for treating vitiligo are usually isolated from the patient and expanded during in vitro culture before transplanted back to the patient. Since melanocytes are naturally adherent cells they are cultured on attaching substratum. There are three potential ways to carry the in vitro expanded melanocytes to the vitiligo lesion; (1) transferring melanocytes and the attaching substratum together to the recipient site. However, this method requires developing a biocompatible/biodegradable cell attaching substratum that can be conveniently transferred from the cell culture vessel to the recipient site, which is more difficult. (2) removing melanocytes from the attaching substratum first, then mixing the cells with hydrogel before transferring them to the recipient site. This method is more expensive and needs an additional process. (3) removing melanocytes from the attaching substratum then mixing them with a biocompatible solution, i.e. preparing a cell suspension, and transferring the suspension to the recipient site. This method is more economical and widely applied.

However, when melanocytes are prepared as a suspension, they aggregate easily and lose vitality quickly in a very short time. If the aggregated melanocytes are transferred to the lesion to be treated, it might form pigment spots or non-uniform pigmentation. Moreover, when the aggregated melanocytes are transferred to the culture vessel for further culture, the culture efficiency is reduced. Therefore, preparing and storing the melanocytes in a way to prevent them from being aggregating is important.

Cell aggregation involves protein and lipid in the cell membrane. The lipid dynamics is greatly influenced by temperature. It is possible to modulate the melanocyte aggregation by adjusting the storage temperature. Temperature also affects cell viability through other mechanisms. Typically adherent mammalian cells in vitro are cultured at 37° C. on attaching substratum. When the cells are detached from the substratum and stored as a cell suspension, the cells may undergo apoptosis and death. In order to increase survival rate of the cells in suspension, it is possible to slow down the cellular chemical reaction or metabolism by lowering the storage temperature. However, low temperature may also cause cold shock and damage to the cells. Although cryo condition is used for long-term storage of cells, the cells ready for being used for clinical treatment are usually stored at room temperature which is usually 25° C. but can vary significantly. Since cells are very sensitive to the temperature, the room temperature might not be the best temperature for storing all cells. Study of the temperature influence on the non-cryo storage of cell suspension is very limited. Particularly there is no related study for melanocytes in this aspect.

For the aforementioned reasons, there is a need to identify a temperature range in which the melanocytes in suspension can sustain good viability as well as being prevented from aggregating.

### SUMMARY

A method is provided for storing melanocyte suspension. First, a melanocyte suspension is prepared. Next, the melanocyte suspension is stored at a temperature ranging between 4-22.5° C.

According to one embodiment of the present invention, the cells in the melanocyte suspension are adult or baby melanocytes, wherein the temperature ranges for storing adult melanocyte suspension is between 10-22.5° C. The temperature range for storing baby melanocyte suspension is between 4-15° C.

According to one embodiment of the present invention, the melanocyte suspension is prepared by Ham's F12 medium, Dulbecco's Modified Eagle medium (DMEM), minimal essential medium (MEM), phosphate buffered saline (PBS), Roswell Park Memorial Institute 1640 medium (RPMI 1640 medium), normal saline or other biocompatible solutions.

In light of the foregoing, the invention allows storing melanocytes as a suspension at low temperature without freezing. In addition, the temperature range of storing melanocyte enables melanocytes to maintain good cell viability and to be prevented from aggregating.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrates a flow chart of an experiment of storing melanocytes according to one embodiment of the present invention;

FIG. 2 illustrates a flow chart of preparing a melanocyte suspension according to one embodiment of the present invention;

FIG. 3 illustrates a flow chart of an experiment of storing baby melanocytes according to one embodiment of the present invention;

FIG. 4 illustrates a flow chart of an experiment of storing adult melanocytes according to one embodiment of the present invention;

FIG. 5 illustrates a statistics bar chart of an experiment of storing baby melanocytes according to one embodiment of the present invention;

FIG. 6 illustrates a statistics bar chart of an experiment of storing adult melanocytes according to one embodiment of the present invention;

FIGS. 7A-7G illustrate the aggregation phenomena of adult melanocytes stored in Ham's F12 solution at different temperatures according to one embodiment of the present invention;

FIGS. 8A-8D illustrate the aggregation phenomena of adult melanocytes stored in DMEM solution at different temperatures according to one embodiment of the present invention;

FIGS. 9A-9D illustrate the aggregation phenomena of adult melanocytes stored in MEM solution at different temperatures according to one embodiment of the present invention;

FIGS. 10A-10D illustrate the aggregation phenomena of adult melanocytes stored in PBS solution at different temperatures according to one embodiment of the present invention; and

FIGS. 11A-11D illustrate the aggregation phenomena of adult melanocytes stored in RPMI medium 1640 solution at different temperatures according to one embodiment of the present invention.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

Experimental Process

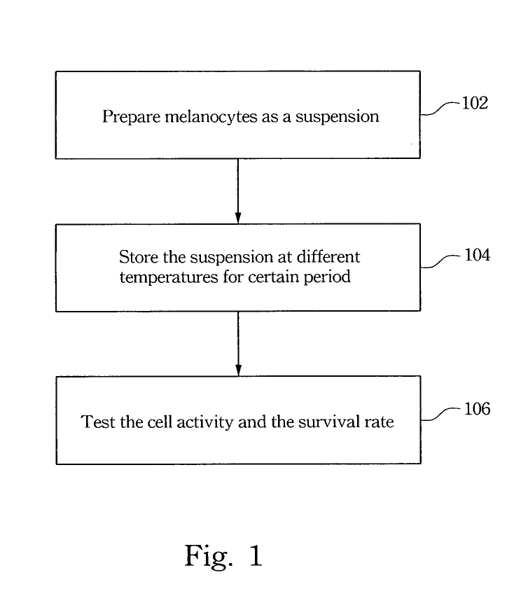

FIG. 1 illustrates the flow chart of one experiment for storing melanocytes according to an embodiment of the present invention. To investigate how the storage temperature and time duration affect the cell survival rate of melanocytes, dermal melanocytes were obtained from baby foreskin or adult epidermis tissue first, and then were cultured and prepared as a suspension (step 102). Next, the suspension was stored under different temperatures for a period of time (step 104). Finally, the cell survival rates were measured (step 106), and cell aggregation was also observed.

In the embodiment of the present invention, the methyl thiazolyl tetrazolium (MTT) method and cell counting were used to measure the cell survival rates. MTT is a conventional method used for measuring the survival rate of cells. The principle of MTT assay is that the viable cells reduce the MTT into dark blue formazan and deposit them in cells by mitochondrial dehydrogenase, which reflects the normal function of mitochondria and cell viability. Then, the cells were lysed with dimethyl sulfoxide (DMSO) to yield the color solution. Finally, the absorbance was measured to quantify the amount of formazan formed. Accordingly, the amount of formazan formed is proportional to the viability of the cells.

Moreover, assessment of cell viability may also be accomplished with detecting viable cells by trypan blue dye exclusion. Since viable cells have intact membranes, they exclude the dye. However, while cell membranes of nonviable cells are damaged, trypan blue can penetrate into cells and convert cells into blue. Hence, nonviable cells can be labeled with the dye. By using a hematocyte counter, viable cells and nonviable cells can be distinguished under the microscope. More detail process and the result of the experiment are as follows.

(A) Preparation of Adult Melanocytes as a Suspension

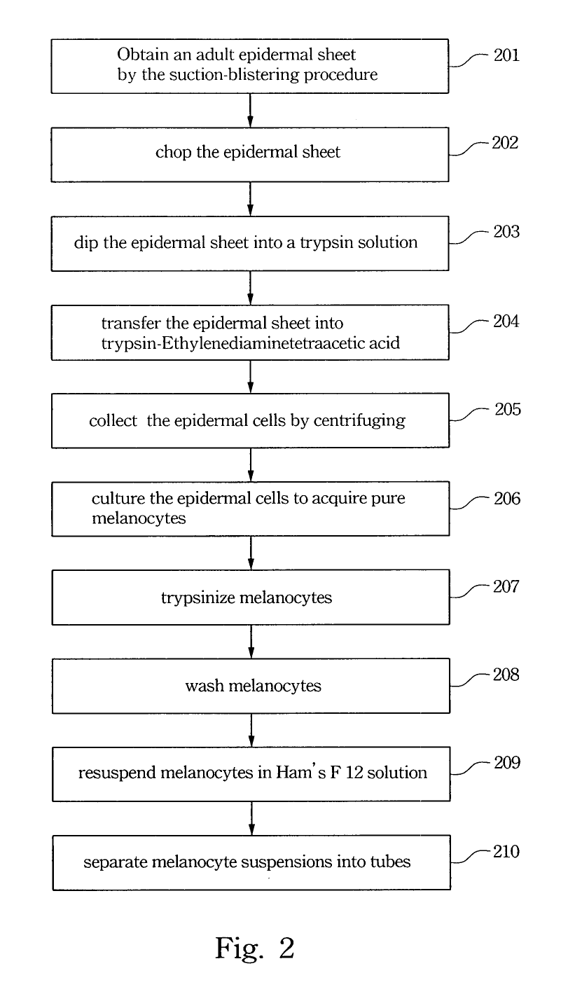

First, referring to FIG. 2, an epidermal sheet was obtained through the suction-blistering procedure without damaging the dermis (step 201). In this procedure, the syringe was attached and fixed to the donor site that has been cleansed with Hibitane. A negative pressure of 200-300 mmHg was applied to the donor site for 1-2 hours, until a dome-shaped blister formed. Then the syringe was removed. The blister was cleansed again with ethanol, and the blister roof was carefully excised and held by a pair of forceps. Generally, there are 86-90% keratocytes and 5-7% melanocytes on the epidermal sheet. Then, the epidermal sheet excised were chopped (step 202), and dipped into trypsin solution (step 203). After that, the chopped epidermal sheet was removed into trypsin-Ethylenediaminetetraacetic acid (trypsin-EDTA) solution to release individual cells (step 204). Finally, the epidermal debris, melanocytes and keratocytes, were collected by centrifuging (step 205), and incubated in the culture dishes (step 206), wherein melanocytes kept growing and keratinocytes could not survive and diminished. Therefore, nearly pure cell colonies of melanocyte were obtained.

In order to detach melanocytes from the culture dishes, trypsin was added to degrade the adherent proteins between melanocytes (step 207). Next, melanocytes obtained were washed (step 208). Then, they were resuspended at a density of 1.2-1.5×10⁶ cells/ml in Ham's F 12 solution (step 209). Ham's F 12 solution is a widely used cell culture and biocompatible medium which comprises rich substances for cell growth, such as amino acids, polysaccharides, fatty acid, and salts, etc. In addition to Ham's F 12 solution, other incubation mediums were also used, for example, Dulbecco's Modified Eagle medium (DMEM), minimal essential medium (MEM), phosphate buffered saline (PBS) and Roswell Park Memorial Institute 1640 medium (RPMI 1640 medium). Finally, the melanocyte suspensions were separated into tubes to proceed to the following experiment (step 210).

(B) An Experiment of Storing Baby Melanocytes

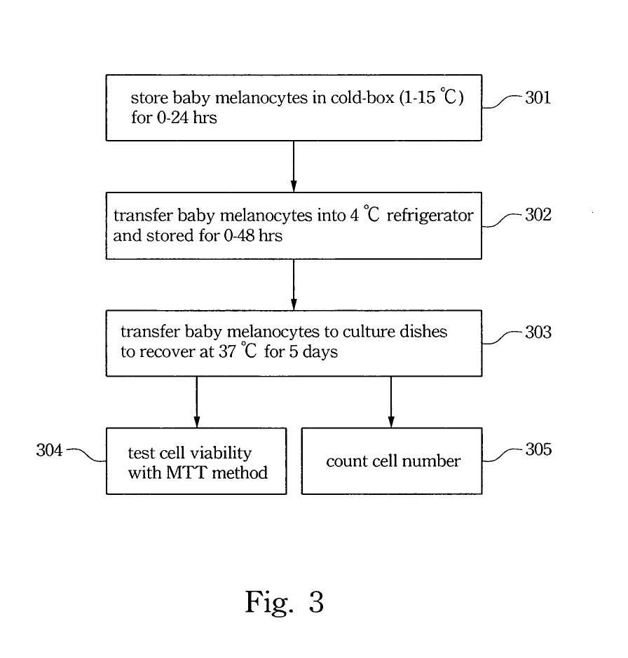

To keep cells at low temperature, the cells can be transported in a portable cold box during transportation then stored in a refrigerator when they arrive to the destination. To simulate this process and to figure out how temperature and time duration affect melanocytes during the process, the melanocytes obtained from babies were processed by two-stage storage. As shown in FIG. 3, the tubes which comprise baby melanocytes were stored in a cold box with a cold-pack from 0 hour (hr) to 24 hrs respectively (step 301). The temperature in this section, 1-15° C., reflected the temperature which may occur during the transportation. In the second section, the tubes in the cold box were transferred to a refrigerator at a temperature of 4° C. and stored from 0 hr to 48 hrs respectively (step 302). This section simulated the storage conditions in a 4° C. refrigerator after arriving at a hospital or an institute. Then, the baby melanocytes were transferred to culture dishes and medium to recover at 37° C. for five days (step 303). Finally, the MTT method (step 304) and cell counting (step 305) were used to measure the cell viability.

In the embodiment of the present invention, table I shows each condition for storing baby melanocytes: (a) analyzed directly before storage; (b) stored at 4° C. for 4 hrs; (c) stored at 4° C. for 24 hrs; (d) stored at 4° C. for 48 hrs; (e) stored in the cold box for 8 hrs first, and then at 4° C. for 16 hrs (total 24 hrs at low temperature); (f) stored in the cold box for 24 hrs; (g) stored in the cold box for 24 hrs first, and then at 4° C. for 24 hrs (total 48 hrs at low temperature).

TABLE I
two-section storage conditions for baby melanocytes

| Sample | Hours stored in Cold boxed | Hours stored in 4° C. | Total |
|---|---|---|---|
| labeled | (1–15° C.) | refrigerator | (hr) |
| a | 0 | 0 | 0 |
| b | 0 | 4 | 4 |
| c | 0 | 24 | 24 |
| d | 0 | 48 | 48 |
| e | 8 | 16 | 24 |
| f | 24 | 0 | 24 |
| g | 24 | 24 | 48 |

(C) An Experiment of Storing Adult Melanocytes

The following experiment was designed to verify the preferred temperature and time duration for storing adult melanocytes.

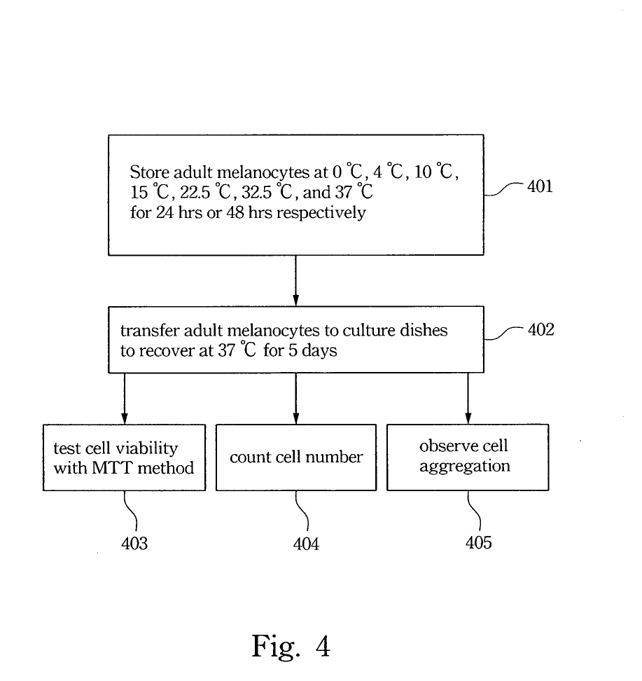

As indicated in FIG. 4, adult melanocytes were stored at 0° C., 4° C., 10° C., 15° C., 22.5° C., 32.5° C., and 37° C. for 24 hrs or 48 hrs respectively (step 401). Next, adult melanocytes were transferred to culture dishes and medium to recover at 37° C. for five days (step 402). Finally, the MTT method (step 403) and cell counting (step 404) were used to measure the cell viability. In addition, cell aggregation was also observed by using microscopes (step 405).

In the embodiment of the present invention, table II shows each condition for storing adult melanocytes: (h) analyzed directly before storage; (i) stored at 4° C. for 24 hrs; (j) stored at 10° C. for 24 hrs; (k) stored at 15° C. for 24 hrs; (l) stored at 22.5° C. for 24 hrs; (m) stored at 32.5° C. for 24 hrs; (n) stored at 37° C. for 24 hrs; (o) stored at 4° C. for 48 hrs; (p) stored at 10° C. for 48 hrs; (q) stored at 15° C. for 48 hrs; (r) stored at 22.5° C. for 48 hrs; (s) stored at 32.5° C. for 48 hrs; (t) stored at 37° C. for 48 hrs.

TABLE II
storage conditions for adult melanocytes

| Sample labeled | Storage temperature (° C.) | Time duration (hr) |
|---|---|---|
| H | N/A | 0 |
| I | 4 | 24 |
| J | 10 | 24 |
| K | 15 | 24 |
| L | 22.5 | 24 |
| M | 32.5 | 24 |
| N | 37 | 24 |
| o | 4 | 48 |
| p | 10 | 48 |
| q | 15 | 48 |
| r | 22.5 | 48 |
| s | 32.5 | 48 |
| t | 37 | 48 |

(D) Results and Discussions

(1) Conditions for Storing Baby Melanocytes

Table III shows the experiment results of cell viability of baby melanocytes that were stored at different temperatures and time durations.

TABLE III
the experiment results of baby melanocytes

| | | An experiment of storing | | | |
|---|---|---|---|---|---|
| | | baby melanocytes | | | |
| Hours | | MTT | | Cell counting | |
| stored in cold box/4° C. | | Percentage | Standard | Percentage | Standard |
| refrigerator (hrs) | | (%) | Deviation | (%) | Deviation |
| a | 0/0 | 100 | 0 | 100 | 0 |
| b | 0/4 | 85.45 | 7.66 | 93.01 | 9.85 |
| c | 0/24 | 103.43 | 7.93 | 82.09 | 21.75 |
| d | 0/48 | 103.55 | 29.72 | 86.26 | 29.57 |
| e | 8/16 | 97.77 | 4.40 | 79.51 | 27.13 |
| f | 24/0 | 92.27 | 11.97 | 75.21 | 28.66 |
| g | 24/24 | 95.87 | 25.54 | 71.21 | 19.18 |

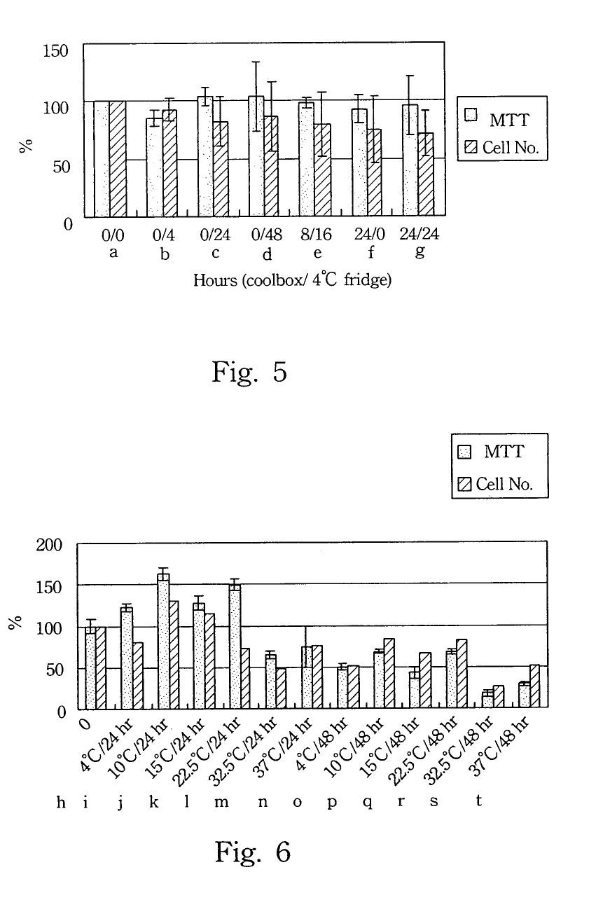

FIG. 5 is the statistical bar chart of table III. The bar with dot indicates the viability measured by MTT, and the bar with oblique line shows the viable cell numbers measured by trypan blue dye exclusion. The values of the samples underwent different storage conditions were correlated with that of the sample before storage which was designated as 100%.

FIG. 5, (a) to (d) suggest that baby melanocytes stored at 4° C. for 0-48 hrs sustained 82% or above viability compared with cells before storage. Thus, baby melanocytes can be stored at 4° C. for almost 48 hrs as a suspension till needed. Moreover, (e) to (g) in FIG. 5 suggest that baby melanocytes stored at between 1 to 15° C. for 8 to 24 hours, and then at 4° C. for up to 24 hours sustained 71% or above viability compared with cells before storage

These results indicate that baby melanocyte suspensions can be stored at 4-15° C. for up to 48 hours and still sustain good viability.

(2) Conditions for Storing Adult Melanocytes

In the experiment (C) the preferred temperature and time duration for storing adult melanocytes was studied and the results were shown in Table IV.

TABLE IV
the experiment results of adult melanocytes

| | | An Experiment of Storing | | | |
|---|---|---|---|---|---|
| | | Adult Melanocytes | | | |
| | | MTT | | Cell Counting | |
| | | Percentage | Standard | | Percentage |
| Temperature/time (hr/° C.) | | (%) | Deviation | Average | (%) |
| h | pre-storage | 100 | 7.96 | 115000 | 100 |
| i | 4/24 | 122.22 | 5.17 | 92500 | 80.43 |
| j | 10/24 | 162.72 | 8.38 | 150000 | 130.43 |
| k | 15/24 | 127.19 | 8.40 | 131250 | 114.13 |
| l | 22.5/24 | 148.87 | 6.39 | 83125 | 72.28 |
| m | 32.5/24 | 65.51 | 4.58 | 54375 | 47.28 |
| n | 37/24 | 74.28 | 25.06 | 87500 | 76.08 |
| o | 4/48 | 50.30 | 4.33 | 59375 | 51.63 |
| p | 10/48 | 68.77 | 2.12 | 95625 | 83.15 |
| q | 15/48 | 43.15 | 7.56 | 77500 | 67.39 |
| r | 22.5/48 | 68.45 | 2.78 | 93750 | 81.52 |
| s | 32.5/48 | 18.65 | 3.40 | 30000 | 26.08 |
| t | 37/48 | 28.96 | 2.04 | 60000 | 52.17 |

FIG. 6 is the statistical bar chart of table IV. FIG. 6 shows the viability of adult melanocytes after being stored at 0-37° C. for 24 hrs or 48 hrs. In FIG. 6, (h) to (n) are representative results of adult melanocytes stored at 0° C., 4° C., 10° C., 15° C., 22.5° C., 32.5° C., and 37° C. for 24 hrs respectively, and (o) to (t) are representative results of adult melanocytes stored at 4° C., 10° C., 15° C., 22.5° C., 32.5° C., and 37° C. for 48 hrs respectively. The values of the samples underwent different storage conditions were correlated with that of the sample before storage which was designated as 100%.

The results indicated that adult melanocytes stored at 10-22.5° C. sustained higher viability. The storage temperatures lower or higher than this range resulted in lower viability. Adult melanocytes stored at 10-22.5° C. for 24 hrs (FIG. 6, (j), (k), (l)) can maintain at least 72% cell viability. However, 48 hrs of storage resulted in decreased cell viability compared with that of 24 hours of storage.

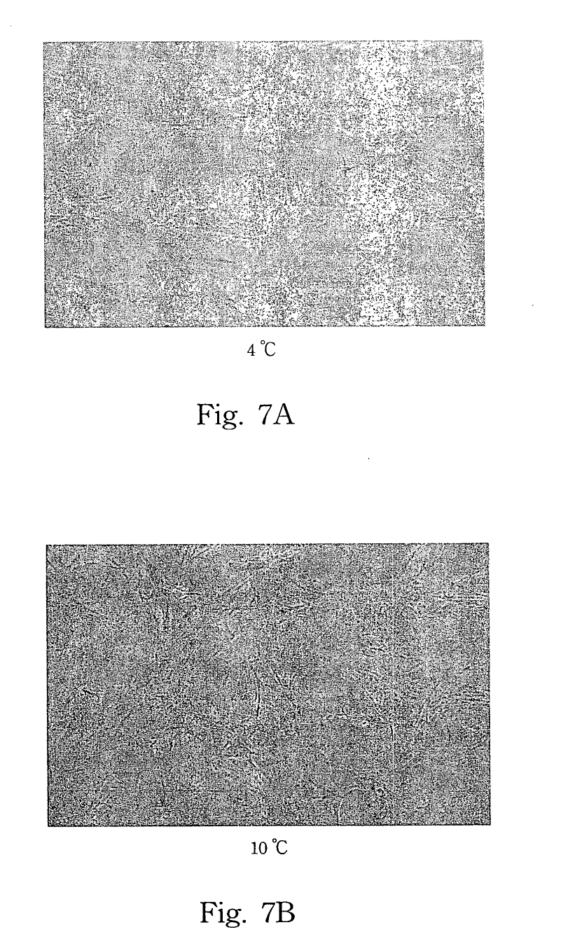

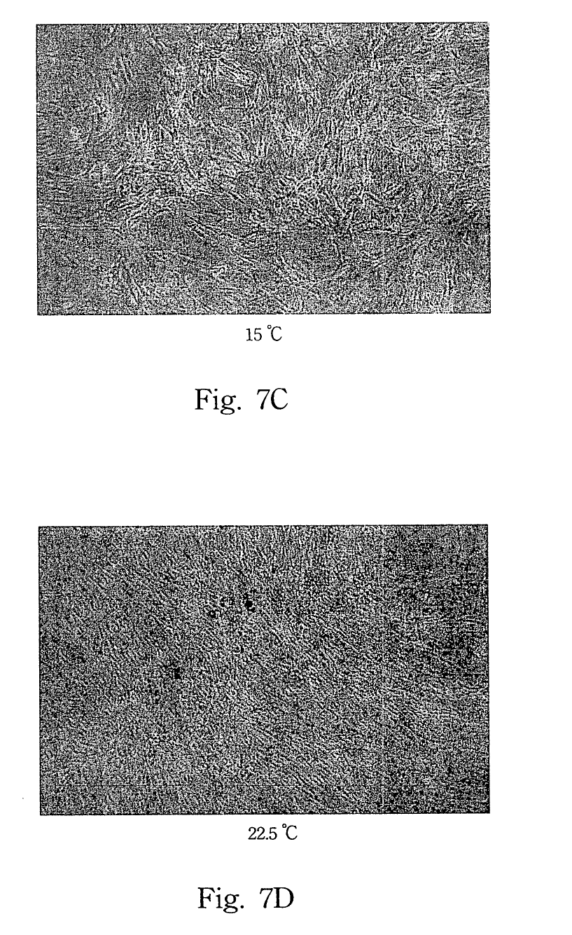

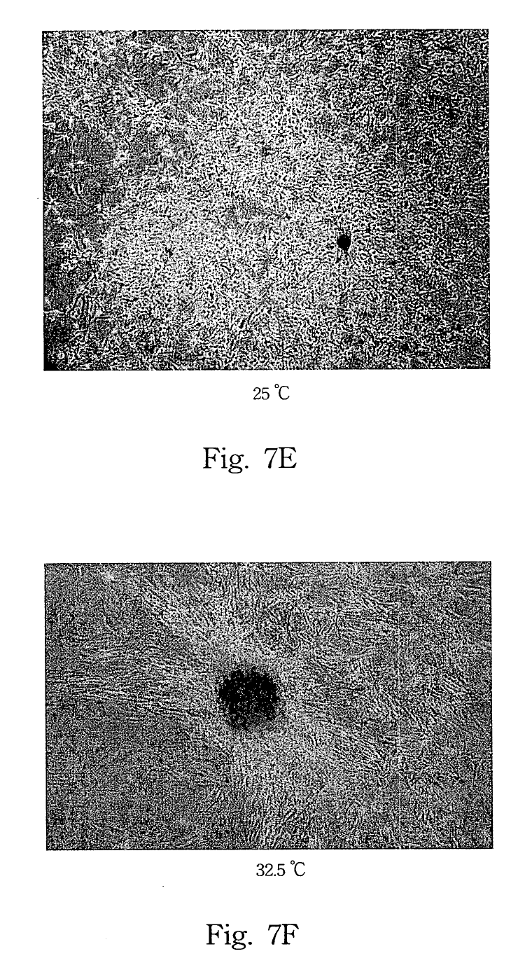

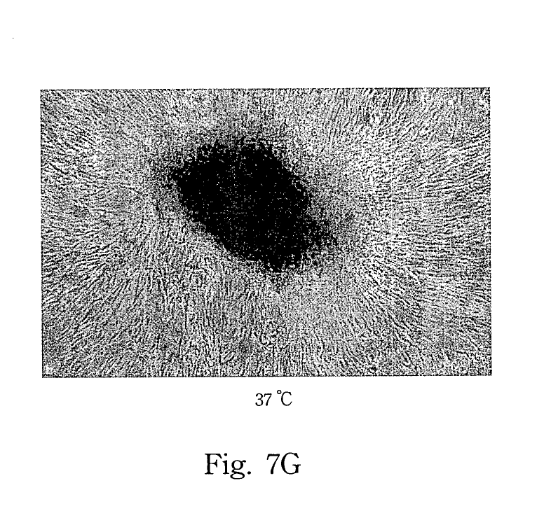

FIGS. 7A-7G illustrate the aggregation conditions of adult melanocytes stored in Ham's F 12 solution at 4° C., 10° C., 15° C., 22.5° C., 32.5° C., and 37° C. for 24 hrs respectively. As shown in FIG. 7A to 7C, adult melanocytes stored at 15° C. or below did not aggregate. Storage at 22.5° C., resulted in slight aggregation (7D). As temperature increased to 32.5° C. and 37° C., the cells aggregated prominently (7E and 7F).

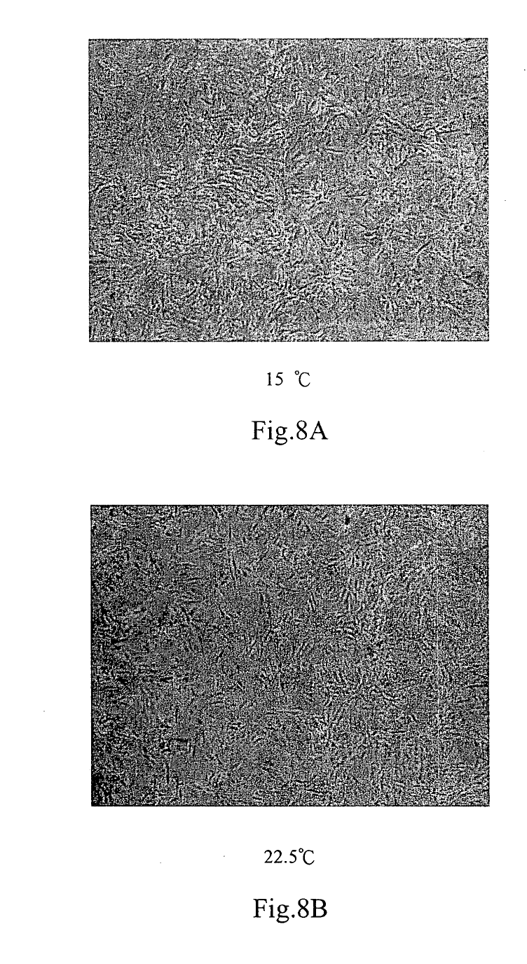

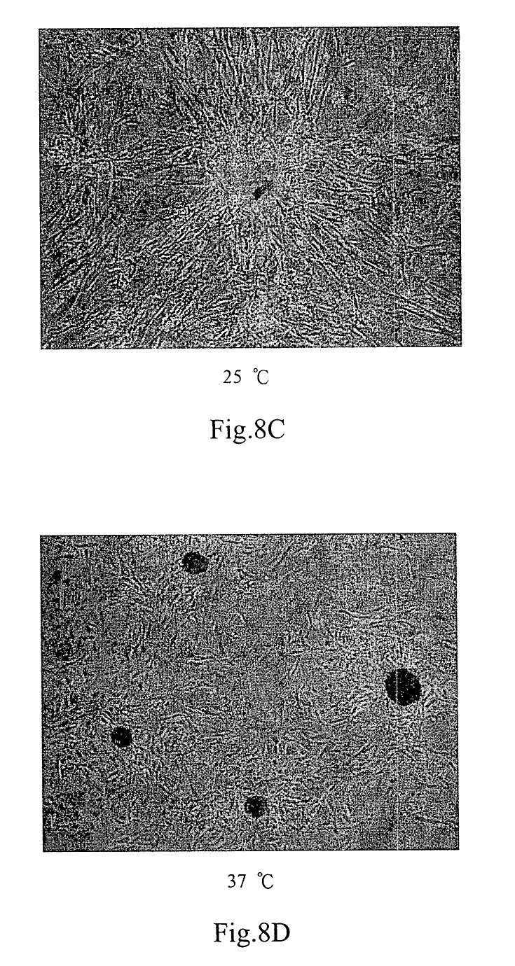

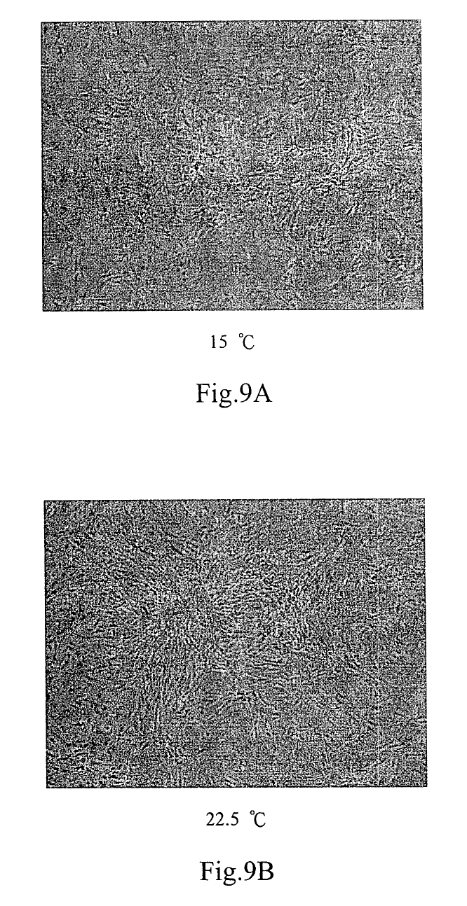

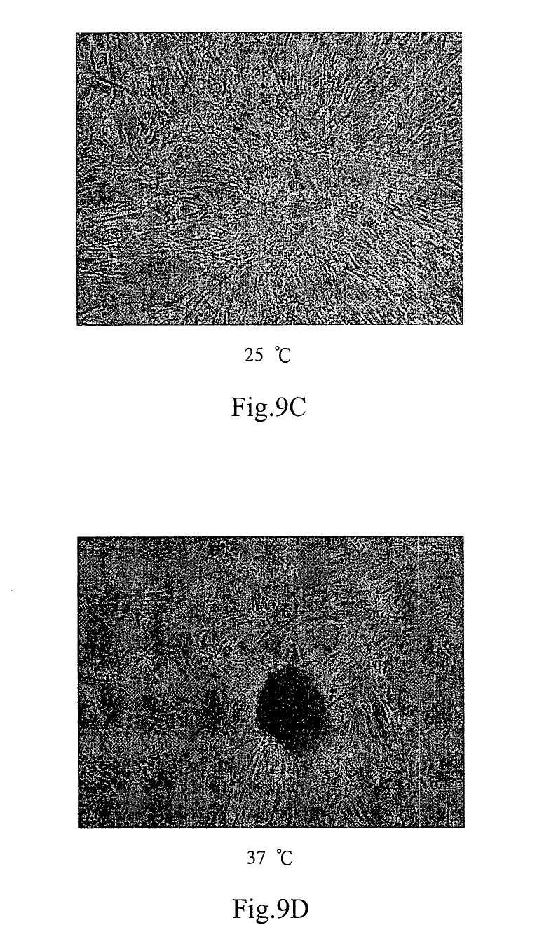

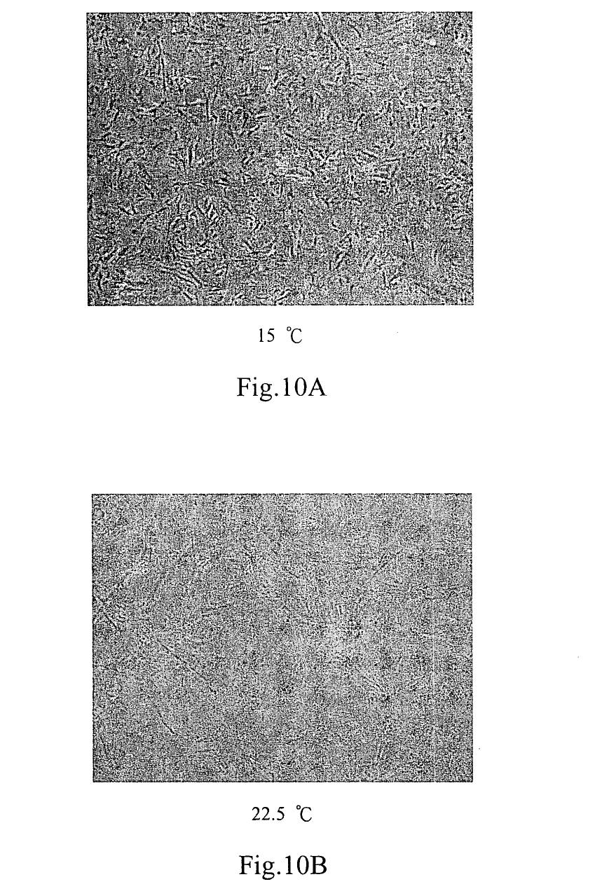

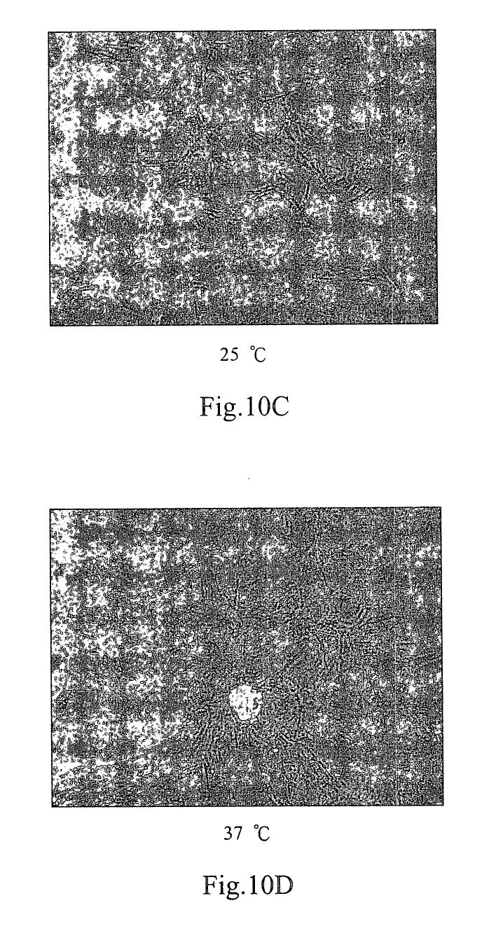

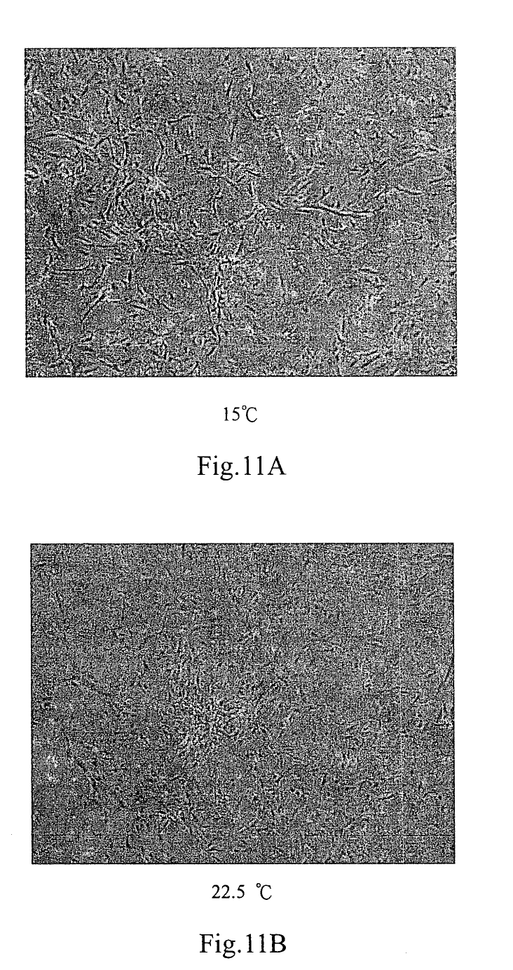

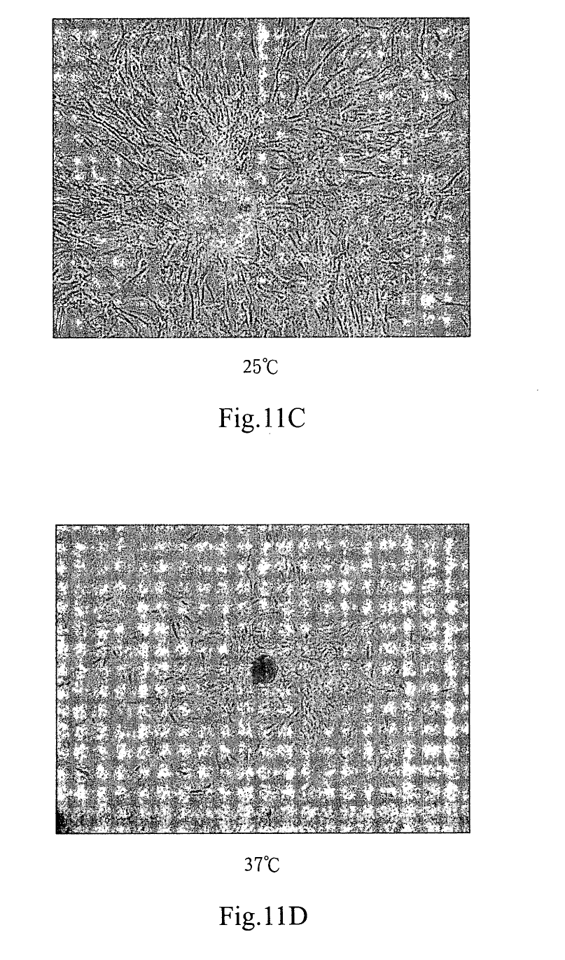

Moreover, it was tested whether the superiority of the storage temperature range can be applied to the melanocytes stored in other biocompatible solutions. In the embodiment of the present invention, melanocytes were stored in four different biocompatible solutions at 15° C., 22.5° C., 25° C., and 37° C. for 24 hrs respectively. Then, they were plated in culture dishes for two days and photographed. These four biocompatible solutions were DMEM, MEM, PBS, and RPMI medium 1640. The experiment results are shown from 15° C., 22.5° C., 25° C., to 37° C. in FIGS. 8A-8D, 9A-9D, 10A-10D, and 11A-11D respectively. As shown in the figures, there was no aggregation at 15° C. while slight aggregation occurred at 22.5° C. As temperature increased to 25° C. and 37° C., the aggregations were prominent.

Judging by the result of cell viability and degree of aggregation, the appropriate temperature range for storing adult melanocytes suspension is 10-22.5° C. The melanocytes stored for 24 hours in this range of temperature sustained at least 72% of viability and the cells remained well segregated.

It is possible to use melanocyte transplantation to improve clinical symptoms resulted from the deficiency of pigmentation, such as all types of leukoderma or gray hairs. The application requires appropriate storage of melanocytes. The present invention enables stored melanocytes to sustain high viability and prevent cell aggregation, therefore enhances efficiency and homogeneity of pigmentation in the treated site.

What is claimed is:

1. A method of storing melanocyte suspension, characterized by not being stored at room temperature (25° C.), which comprises:
preparing a melanocyte suspension; and
storing the melanocyte suspension at a temperature range between 4-22.5° C.

2. The method of claim 1, wherein the melanocyte suspension is prepared by Ham's F12 medium, Dulbecco's Modified Eagle medium (DMEM), minimal essential medium (MEM), phosphate buffered saline (PBS), Roswell Park Memorial Institute 1640 medium (RPMI 1640 medium), normal saline or other biocompatible solutions.

3. The method of claim 1, wherein melanocytes used for preparing the melanocyte suspension are adult melanocytes.

4. The method of claim 3, wherein the temperature range is between 10-22.5° C.

5. The method of claim 1, wherein melanocytes used for preparing the melanocyte suspension are baby melanocytes.

6. The method of claim 5, wherein the temperature range is between 4-15° C.

7. A method of storing adult melanocyte suspension, characterized by not being stored at room temperature (25° C.), which comprises:
preparing an adult melanocyte suspension, wherein the adult melanocyte suspension is prepared by Dulbecco's Modified Eagle medium (DMEM), minimal essential medium (MEM), phosphate buffered saline (PBS), Roswell Park Memorial Institute 1640 medium (RPMI 1640 medium), normal saline or other biocompatible solutions; and
storing the adult melanocyte suspension at a temperature range between 10-22.5° C.

8. A method of storing baby melanocyte suspension, characterized by not being stored at room temperature (25° C.), which comprises:
preparing a baby melanocyte suspension, wherein the baby melanocyte suspension is prepared by Dulbecco's Modified Eagle medium (DMEM), minimal essential medium (MEM), phosphate buffered saline (PBS), Roswell Park Memorial Institute 1640 medium (RPMI 1640 medium), normal saline or other biocompatible solutions; and
storing the baby melanocyte suspension at a temperature range between 4-15° C.
